(19) United States
(12) Patent Application Publication
(10) Pub. No.: US 2017/0086789 A1
(43) Pub. Date: Mar. 30, 2017

(54) METHODS AND SYSTEMS FOR PROVIDING A MEAN VELOCITY

(71) Applicant: General Electric Company, Schenectady, NY (US)

(72) Inventors: Helmut Brandl, Zipf (AT); Josef Steininger, Zipf (AT)

(21) Appl. No.: 14/871,167

(22) Filed: Sep. 30, 2015

(51) Int. Cl. A61B 8/00; A61B 8/14; A61B 8/08; G01S 7/52; A61B 8/06

(52) U.S. Cl. CPC A61B 8/463; G01S 7/52085; A61B 8/06; A61B 8/469; A61B 8/5223; A61B 8/14; A61B 8/4444; A61B 8/4427; A61B 8/565; A61B 8/4405; A61B 8/54; A61B 8/488

(57) ABSTRACT

Systems and methods for providing a mean velocity are provided. The systems and methods collect ultrasound data over a period of time for a volumetric region of interest (ROI). The systems and methods display a color flow image based on the ultrasound data, and designate a spatial gate on the color flow image. The spatial gate corresponding to a set of voxels adjacent to one another and within the color flow image. The systems and methods further calculate a mean velocity associated with the set of voxels at a select time, and repeat the calculate operation for multiple select times over the period of time to derive a series of mean velocities. The systems and methods also present indicia indicative of the series of mean velocities exhibited by the set of voxels within the spatial gate over the period of time.

### FIELD

Embodiments described herein generally relate to providing a mean velocity on a diagnostic medical imaging system based on ultrasound data.

### BACKGROUND OF THE INVENTION

Diagnostic medical imaging systems typically include a scan portion and a control portion having a display. For example, ultrasound imaging systems usually include ultrasound scanning devices, such as ultrasound probes having transducers that are connected to an ultrasound system to control the acquisition of ultrasound data by performing various ultrasound scans (e.g., imaging a volume or body). The ultrasound systems are controllable to operate in different modes of operation to perform the different scans. The signals received at the probe are then communicated and processed at a back end. When the scan is complete, the ultrasound data may be stored on a patient archive communication system (PACS) for retrospective examination.

Conventional ultrasound imaging systems include a set of imaging modes, such as B-mode, color flow, and spectral Doppler imaging. In the B-mode, such ultrasound imaging systems create two-dimensional images of tissue in which the brightness of a pixel is based on the intensity of the echo return. Alternatively, in a color flow imaging mode, the general movement or velocity of fluid (e.g., blood) or tissue is imaged using different colors to represent speed and direction of flow.

However, to perform velocity measurements spectral Doppler imaging is used. In conventional ultrasound spectral Doppler imaging, the operator is required to manually position a sample gate to a measurement location in a two-dimensional image with or without color flow data. The operator also needs to manually adjust the sample gate size relative to the diameter of the vessel to be studied. From the acoustic data acquired over many transmit firings, Doppler frequency spectral data is obtained via standard Fast Fourier Transform (FFT) spectral analysis. If the spectral Doppler imaging was not performed during the scan, velocity measurements are not retrievable from the PACS during retrospective examination. Thereby limiting the use of a retrospective audit and/or review of previous diagnosis and/or measurements.

### BRIEF DESCRIPTION OF THE INVENTION

In one embodiment, a method for providing a mean velocity for ultrasound data is provided. The method may include collecting ultrasound data over a period of time for a volumetric region of interest. The method may further include displaying a color flow image based on the ultrasound image, and designated a spatial gate on the color flow image. The spatial gate corresponding to a set of voxels adjacent to one another and within the color flow image. The method may also include calculating a mean velocity associated with the set of voxels at a select time, and repeating the calculating operation for multiple select times over the period of time to derive a series of mean velocities. The method may further present indicia indicative of the series of mean velocities exhibited by the set of voxels within the spatial gate over the period of time.

In another embodiment, an ultrasound imaging system is provided. The ultrasound imaging system may include an ultrasound probe configured to acquire ultrasound imaging data of a patient, and a display. The ultrasound imaging system may also include a memory configured to store programmed instructions, and one or more processors configured to execute the programmed instructions stored in the memory. The one or more processors when executing the programmed instructions perform one or more operations. The one or more processors may collect the ultrasound data from the ultrasound probe over a period of time for a volumetric region of interest, and display a color flow image based on the ultrasound data on the display. The one or more processors may designate a spatial gate on the color flow image. The spatial gate corresponding to a set of voxels adjacent to one another and within the color flow image. The one or more processors may further calculate a mean velocity associated with the set of voxels at a select time, and repeat the calculating operation for multiple select time over the period of time to derive a series of mean velocities. The one or more processors may further present indicia indicative of the series of mean velocities exhibited by the set of voxels within the spatial gate over the period time.

In another embodiment, a tangible and non-transitory computer readable medium may include one or more computer software modules configured to direct one or more processors. The one or more computer software modules may be configured to direct the one or more processors to collect ultrasound data over a period of time for a volumetric region of interest, display a color flow image based on the ultrasound data, and designate a spatial gate on the color flow image. The spatial gate corresponding to a set of voxels adjacent to one another and within the color flow image. The one or more computer software modules may be configured to direct the one or more processors to calculate a mean velocity associated with the set of voxels at a select time, repeat the calculating operation for multiple select times over the period of time to derive a series of mean velocities, and present indicia indicative of the series of mean velocities exhibited by the set of voxels within the spatial gate over the period of time.

### BRIEF DESCRIPTION OF THE DRAWINGS

FIG. 1 illustrates a schematic block diagram of an ultrasound imaging system, in accordance with an embodiment.

FIG. 2 is an illustration of a simplified block diagram of a controller circuit of the ultrasound imaging system of FIG. 1, in accordance with an embodiment.

FIG. 3 illustrate a flowchart of a method for measuring a cardiac output, in accordance with an embodiment.

FIG. 4 illustrates a color flow image of a region of interest based on ultrasound data, in accordance with an embodiment.

FIG. 5 illustrates multiple color flow images of a region of interest based on ultrasound data, in accordance with an embodiment.

FIG. 6 is an indicia corresponding to a graphical representation of a series of mean velocities, in accordance with an embodiment.

FIG. 7 illustrates a 3D capable miniaturized ultrasound system having a probe that may be configured to acquire 3D ultrasonic data or multi-plane ultrasonic data.

FIG. 8 illustrates a hand carried or pocket-sized ultrasound imaging system wherein the display and user interface form a single unit.

FIG. 9 illustrates an ultrasound imaging system provided on a movable base.

### DETAILED DESCRIPTION OF THE INVENTION

The following detailed description of certain embodiments will be better understood when read in conjunction with the appended drawings. To the extent that the figures illustrate diagrams of the functional modules of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide systems and methods for presenting a mean velocity of a real-time color flow image (e.g., four dimension, cine), a spatio-temporal image correlation (STIC) color flow image dataset, and/or the like. The mean velocity may be calculated from 4D Doppler volume information, which is used to generate the color flow image over a time period. The mean velocity may correspond to a flow velocity and/or movement of tissue represented by a group of voxels located within a spatial gate position. The spatial gate may be positioned by a user within the color flow image defining the group of voxels within the color flow image. In various embodiments, the mean velocity may be calculated over select times within the time period to form a graphical waveform based on the spatial gate position.

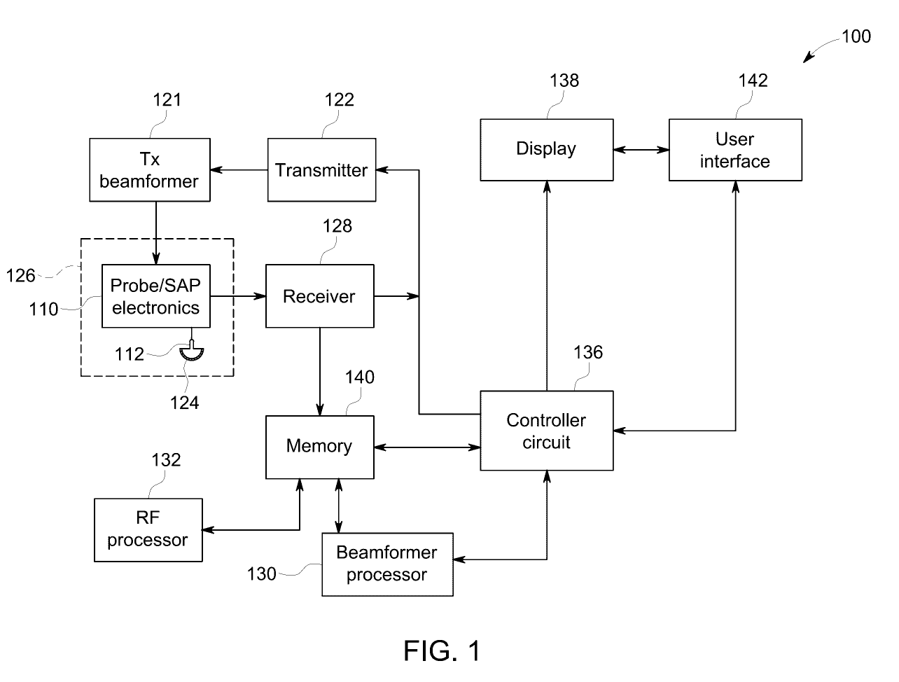

FIG. 1 is a schematic diagram of a diagnostic medical imaging system, specifically, an ultrasound imaging system 100. The ultrasound imaging system 100 includes an ultrasound probe 126 having a transmitter 122 and probe/SAP electronics 110. The ultrasound probe 126 may be configured to acquire ultrasound data or information from a region of interest (e.g., organ, blood vessel) of the patient. The ultrasound probe 126 is communicatively coupled to the controller circuit 136 via the transmitter 122. The transmitter 122 transmits a signal to a transmit beamformer 121 based on acquisition settings received by the user. The signal transmitted by the transmitter 122 in turn drives the transducer elements 124 within the transducer array 112. The transducer elements 124 emit pulsed ultrasonic signals into a patient (e.g., a body). A variety of a geometries and configurations may be used for the array 112. Further, the array 112 of transducer elements 124 may be provided as part of, for example, different types of ultrasound probes.

The acquisition settings may define an amplitude, pulse width, frequency, and/or the like of the ultrasonic pulses emitted by the transducer elements 124. The acquisition settings may be adjusted by the user by selecting a gain setting, power, time gain compensation (TGC), resolution, and/or the like from the user interface 142.

The transducer elements 124, for example piezoelectric crystals, emit pulsed ultrasonic signals into a body (e.g., patient) or volume corresponding to the acquisition settings. The ultrasonic signals may include, for example, one or more reference pulses, one or more pushing pulses (e.g., shear-waves), and/or one or more pulsed wave Doppler pulses. At least a portion of the pulsed ultrasonic signals backscatter from a region of interest (ROI) (e.g., heart, left ventricular outflow tract, breast tissues, liver tissues, cardiac tissues, prostate tissues, and the like) to produce echoes. The echoes are delayed in time and/or frequency according to a depth or movement, and are received by the transducer elements 124 within the transducer array 112. The ultrasonic signals may be used for imaging, for generating and/or tracking shear-waves, for measuring changes in position or velocity within the ROI (e.g., movement of blood cells), differences in compression displacement of the tissue (e.g., strain), and/or for therapy, among other uses. For example, the probe 126 may deliver low energy pulses during imaging and tracking, medium to high energy pulses to generate shear-waves, and high energy pulses during therapy.

The transducer array 112 may have a variety of array geometries and configurations for the transducer elements 124 which may be provided as part of, for example, different types of ultrasound probes 126. The probe/SAP electronics 110 may be used to control the switching of the transducer elements 124. The probe/SAP electronics 110 may also be used to group the transducer elements 124 into one or more sub-apertures.

The transducer elements 124 convert the received echo signals into electrical signals which may be received by a receiver 128. The receiver 128 may include one or more amplifiers, an analog to digital converter (ADC), and/or the like. The receiver 128 may be configured to amplify the received echo signals after proper gain compensation and convert these received analog signals from each transducer element 124 to digitized signals sampled uniformly in time. The digitized signals representing the received echoes are stored on memory 140, temporarily. The digitized signals correspond to the backscattered waves receives by each transducer element 124 at various times. After digitization, the signals still may preserve the amplitude, frequency, phase information of the backscatter waves.

Optionally, the controller circuit 136 may retrieve the digitized signals stored on the memory 140 to prepare for the beamformer processor 130. For example, the controller circuit 136 may convert the digitized signals to baseband signals or compressing the digitized signals.

The beamformer processor 130 may include one or more processors. Optionally, the beamformer processor 130 may include a central controller circuit (CPU), one or more microprocessors, or any other electronic component capable of processing inputted data according to specific logical instructions. Additionally or alternatively, the beamformer processor 130 may execute instructions stored on a tangible and non-transitory computer readable medium (e.g., the memory 140) for beamforming calculations using any suitable beamforming method such as adaptive beamforming, synthetic transmit focus, aberration correction, synthetic aperture, clutter reduction and/or adaptive noise control, and/or the like.

The beamformer processor 130 may further perform filtering and decimation, such that only the digitized signals corresponding to relevant signal bandwidth is used, prior to beamforming of the digitized data. For example, the beamformer processor 130 may form packets of the digitized data based on scanning parameters corresponding to focal zones, expanding aperture, imaging mode (B-mode, color flow), and/or the like. The scanning parameters may define channels and time slots of the digitized data that may be beamformed, with the remaining channels or time slots of digitized data that may not be communicated for processing (e.g., discarded).

The beamformer processor 130 performs beamforming on the digitized signals and outputs a radio frequency (RF) signal. The RF signal is then provided to an RF processor 132 that processes the RF signal. The RF processor 132 may generate different ultrasound image data types, e.g. B-mode, color Doppler (velocity/power/variance), tissue Doppler (velocity), and Doppler energy, for multiple scan planes or different scanning patterns. For example, the RF processor 132 may generate tissue Doppler data for multi-scan planes. The RF processor 132 gathers the information (e.g. I/Q, B-mode, color Doppler, tissue Doppler, and Doppler energy information) related to multiple data slices and stores the data information, which may include time stamp and orientation/rotation information, on the memory 140.

Alternatively, the RF processor 132 may include a complex demodulator (not shown) that demodulates the RF signal to form IQ data pairs representative of the echo signals. The RF or IQ signal data may then be provided directly to the memory 140 for storage (e.g., temporary storage). Optionally, the output of the beamformer processor 130 may be passed directly to the controller circuit 136.

The controller circuit 136 may be configured to process the acquired ultrasound data (e.g., RF signal data or IQ data pairs) and prepare frames of ultrasound image data for display on the display 138. The controller circuit 136 may include one or more processors. Optionally, the controller circuit 136 may include a central controller circuit (CPU), one or more microprocessors, a graphics controller circuit (GPU), or any other electronic component capable of processing inputted data according to specific logical instructions. Having the controller circuit 136 that includes a GPU may be advantageous for computation-intensive operations, such as volume-rendering. Additionally or alternatively, the controller circuit 136 may execute instructions stored on a tangible and non-transitory computer readable medium (e.g., the memory 140).

The controller circuit 136 is configured to perform one or more processing operations according to a plurality of selectable ultrasound modalities on the acquired ultrasound data, adjust or define the ultrasonic pulses emitted from the transducer elements 124, adjust one or more image display settings of components (e.g., ultrasound images, interface components, positioning regions of interest) displayed on the display 138, and other operations as described herein. Acquired ultrasound data may be processed in real-time by the controller circuit 136 during a scanning or therapy session as the echo signals are received. Additionally or alternatively, the ultrasound data may be stored temporarily on the memory 140 during a scanning session and processed in less than real-time in a live or off-line operation.

The memory 140 may be used for storing processed frames of acquired ultrasound data that are not scheduled to be displayed immediately or to store post-processed images (e.g., shear-wave images, strain images), firmware or software corresponding to, for example, a graphical user interface, one or more default image display settings, programmed instructions (e.g., for the controller circuit 136, the beamformer processor 130, the RF processor 132), and/or the like. The memory 140 may be a tangible and non-transitory computer readable medium such as flash memory, RAM, ROM, EEPROM, and/or the like.

The memory 140 may store 3D ultrasound image data sets of the ultrasound data, where such 3D ultrasound image data sets are accessed to present 2D and 3D images. For example, a 3D ultrasound image data set may be mapped into the corresponding memory 140, as well as one or more reference planes. The processing of the ultrasound data, including the ultrasound image data sets, may be based in part on user inputs, for example, user selections received at the user interface 142.

The controller circuit 136 is operably coupled to a display 138 and a user interface 142. The display 138 may include one or more liquid crystal displays (e.g., light emitting diode (LED) backlight), organic light emitting diode (OLED) displays, plasma displays, CRT displays, and/or the like. The display 138 may display patient information, ultrasound images and/or videos, components of a display interface, one or more 2D, 3D, or 4D ultrasound image data sets from ultrasound data stored on the memory 140 or currently being acquired, measurements, diagnosis, treatment information, and/or the like received by the display 138 from the controller circuit 136.

The user interface 142 controls operations of the controller circuit 136 and is configured to receive inputs from the user. The user interface 142 may include a keyboard, a mouse, a touchpad, one or more physical buttons, and/or the like. Optionally, the display 138 may be a touch screen display, which includes at least a portion of the user interface 142.

For example, a portion of the user interface 142 may correspond to a graphical user interface (GUI) generated by the controller circuit 136 shown on the display. The GUI may include one or more interface components that may be selected, manipulated, and/or activated by the user operating the user interface 142 (e.g., touch screen, keyboard, mouse). The interface components may be presented in varying shapes and colors, such as a graphical or selectable icon, a slide bar, a cursor, and/or the like. Optionally, one or more interface components may include text or symbols, such as a drop-down menu, a toolbar, a menu bar, a title bar, a window (e.g., a pop-up window) and/or the like. Additionally or alternatively, one or more interface components may indicate areas within the GUI for entering or editing information (e.g., patient information, user information, diagnostic information), such as a text box, a text field, and/or the like.

In various embodiments, the interface components may perform various functions when selected, such as measurement functions, editing functions, database access/search functions, diagnostic functions, controlling acquisition settings, and/or system settings for the ultrasound imaging system 100 performed by the controller circuit 136.

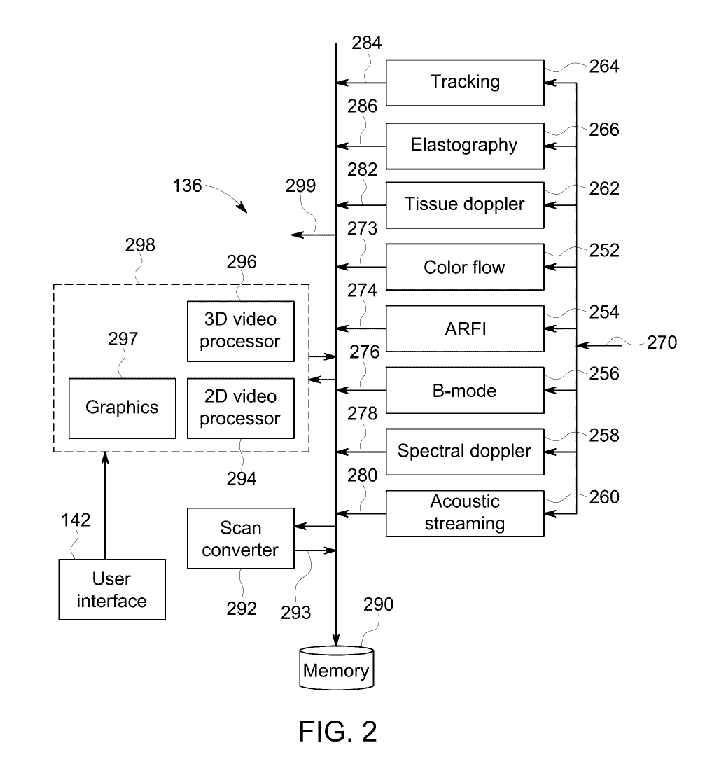

FIG. 2 is an exemplary block diagram of the controller circuit 136. The controller circuit 136 is illustrated in FIG. 2 conceptually as a collection of circuits and/or software modules, but may be implemented utilizing any combination of dedicated hardware boards, DSPs, one or more processors, FPGAs, ASICs, a tangible and non-transitory computer readable medium configured to direct one or more processors, and/or the like.

The circuits 252-266 perform mid-processor operations representing one or more software features of the ultrasound imaging system 100. The controller circuit 136 may receive ultrasound data 270 in one of several forms. In the embodiment of FIG. 1, the received ultrasound data 270 constitutes IQ data pairs representing the real and imaginary components associated with each data sample of the digitized signals. The IQ data pairs are provided to one or more circuits, for example, a color-flow circuit 252, an acoustic radiation force imaging (ARFI) circuit 254, a B-mode circuit 256, a spectral Doppler circuit 258, an acoustic streaming circuit 260, a tissue Doppler circuit 262, a tracking circuit 264, and an electrography circuit 266. Other circuits may be included, such as an M-mode circuit, power Doppler circuit, among others. However, embodiments described herein are not limited to processing IQ data pairs. For example, processing may be done with RF data and/or using other methods. Furthermore, data may be processed through multiple circuits.

Each of circuits 252-266 is configured to process the IQ data pairs in a corresponding manner to generate, respectively, color-flow data 273, ARFI data 274, B-mode data 276, spectral Doppler data 278, acoustic streaming data 280, tissue Doppler data 282, tracking data 284, electrography data 286 (e.g., strain data, shear-wave data), among others, all of which may be stored in a memory 290 (or the memory 140 shown in FIG. 1) temporarily before subsequent processing. The data 273-286 may be stored, for example, as sets of vector data values, where each set defines an individual ultrasound image frame. The vector data values are generally organized based on the polar coordinate system.

A scan converter circuit 292 accesses and obtains from the memory 290 the vector data values associated with an image frame and converts the set of vector data values to Cartesian coordinates to generate an ultrasound image frame 293 formatted for display. The ultrasound image frames 293 generated by the scan converter circuit 292 may be provided back to the memory 290 for subsequent processing or may be provided to the memory 140. Once the scan converter circuit 292 generates the ultrasound image frames 293 associated with the data, the image frames may be stored in the memory 290 or communicated over a bus 299 to a database (not shown), the memory 140, and/or to other processors (not shown).

The display circuit 298 accesses and obtains one or more of the image frames from the memory 290 and/or the memory 140 over the bus 299 to display the images onto the display 138. The display circuit 298 receives user input from the user interface 142 selecting one or image frames to be displayed that are stored on memory (e.g., the memory 290) and/or selecting a display layout or configuration for the image frames.

The display circuit 298 may include a 2D video processor circuit 294. The 2D video processor circuit 294 may be used to combine one or more of the frames generated from the different types of ultrasound information. Successive frames of images may be stored as a cine loop (4D images) in the memory 290 or memory 140. The cine loop represents a first in, first out circular image buffer to capture image data that is displayed in real-time to the user. The user may freeze the cine loop by entering a freeze command at the user interface 142.

The display circuit 298 may include a 3D processor circuit 296. The 3D processor circuit 296 may access the memory 290 to obtain spatially consecutive groups of ultrasound image frames and to generate three-dimensional image representations thereof, such as through volume rendering or surface rendering algorithms as are known. The three-dimensional images may be generated utilizing various imaging techniques, such as ray-casting, maximum intensity pixel or voxel projection and the like.

The display circuit 298 may include a graphic circuit 297. The graphic circuit 297 may access the memory 290 to obtain groups of ultrasound image frames and the ROI data acquisition locations that have been stored or that are currently being acquired. The graphic circuit 297 may generate images that include the images of the ROI and a graphical representation positioned (e.g., overlaid) onto the images of the ROI. The graphical representation may represent an outline of a treatment space, the focal point or region of the therapy beam, a path taken by the focal region within the treatment space, a probe used during the session, the ROI data acquisition location, and the like. Graphical representations may also be used to indicate the progress of the therapy session. The graphical representations may be generated using a saved graphical image or drawing (e.g., computer graphic generated drawing), or the graphical representation may be directly drawn by the user onto the image using a GUI of the user interface 142.

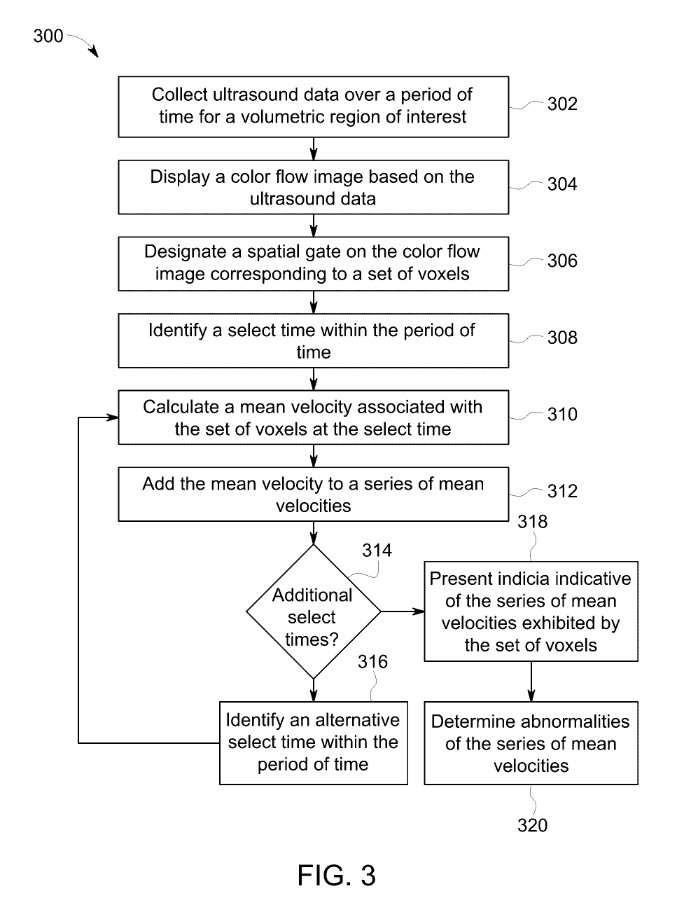

In connection with FIG. 3, the user may select an interface component corresponding to measuring a mean velocity via the user interface 142. When the interface component is selected, the controller circuit 136 may perform one or more of the operations described in connection with method 300.

FIG. 3 illustrate a flowchart of a method 300 for providing a mean velocity, in accordance with various embodiments described herein. The method 300, for example, may employ structures or aspects of various embodiments (e.g., systems and/or methods) discussed herein. In various embodiments, certain steps (or operations) may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 300 may be used as one or more algorithms to direct hardware to perform one or more operations described herein. It should be noted, other methods may be used, in accordance with embodiments herein.

One or more methods may (i) collect ultrasound data over a period of time for a volumetric region of interest, (ii) display a color flow image based on the ultrasound data, (iii) designate a spatial gat on the color flow image, (iv) calculate a mean velocity associated with the set of voxels at a select time, (v) repeat the calculation operation for multiple select times over the period of time to derive a series of mean velocities, and (vi) present indicia indicative of the series of mean velocities exhibited by the set of voxels within the spatial gate over the period of time.

Beginning at 302, the controller circuit 136 may collect ultrasound data over a period of time for a volumetric region of interest (ROI). The ROI may correspond to a cardiac structure, such as a heart, left ventricle, right ventricle, left ventricular outflow tract, and/or the like. The user may position the ultrasound probe 126 (FIG. 1) to align the transducer array 112 to, for example, an abdominal view, a four-chamber, five-chamber, short-axis and three-vessel view of the heart.

The ultrasound acquisition settings for collecting ultrasound data by the ultrasound probe 126 may be defined based on a selection by the user. For example, based on a selection of an interface component and/or select keystroke corresponding to one or more of the circuits 260-266, by the user using the user interface 142 (FIG. 1).

The ultrasound data may include Doppler data. For example, the user may select a color flow mode acquisition interface component, which instructs the controller circuit 136 to configure the ultrasound acquisition settings (e.g., the gain, power, time gain compensation (TGC), resolution, and/or the like of the ultrasound probe 126 of the ultrasound probe 126) and process received ultrasound imaging data. Based on the ultrasound acquisition settings, the transducer elements 124 emit ultrasonic pulses over the period of time, of which, at least a portion are backscattered by the tissue with a corresponding phase shift and received by the transducer elements 124. The controller circuit 136 receives the ultrasound data 270, which includes the phase shift information (e.g., Doppler data).

Additionally or alternatively, ultrasound data may be collected from the memory 290, the memory 140, and/or remotely from a PACS server (not shown). For example, the memory 290 or 140 and/or PACS server may store the medical images (e.g., color flow ultrasound images) and corresponding ultrasound data acquired over the period of time from previous scans of the ultrasound imaging system 100 within a database or registry corresponding to an electronic medical record of the patient. The user may access and/or load the ultrasound data from the memory 290, the memory 140, the PACS server, and/or the like by selecting the ultrasound data via the user interface 142, which may correspond to a retrospective examination

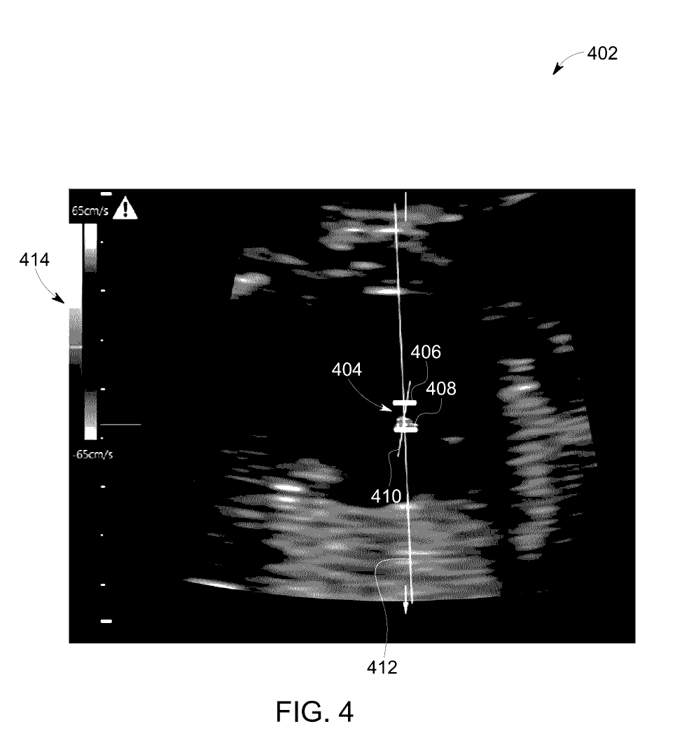

At 304, the controller circuit 136 displays a color flow image 402 based on the ultrasound data. FIG. 4 illustrates the color flow image 402, which may be shown on the display 138. The color flow image 402 may correspond to one of the frames generated by the controller circuit 136 from a plurality of color flow image frames collected during the period of time. For example, the controller circuit 136 may calculate a velocity (e.g., a flow velocity) of the tissue from the Doppler data (e.g., the phase shift) of the ultrasound data with respect to the ultrasound probe 126 to generate the color flow data 273. The velocity information may be included within vector data values of the color flow data 273 defining individual frames of the color flow image 402, which may be stored in the memory 290. The vector data values may include voxel color information, such as red and blue, to represent a speed and a direction (e.g., with respect to the ultrasound probe 126) of the flow. For example, a color meter 414 with a defined color spectrum is shown with the color flow image 402, which relates or associates a color with a corresponding speed (e.g., cm/s) and direction.

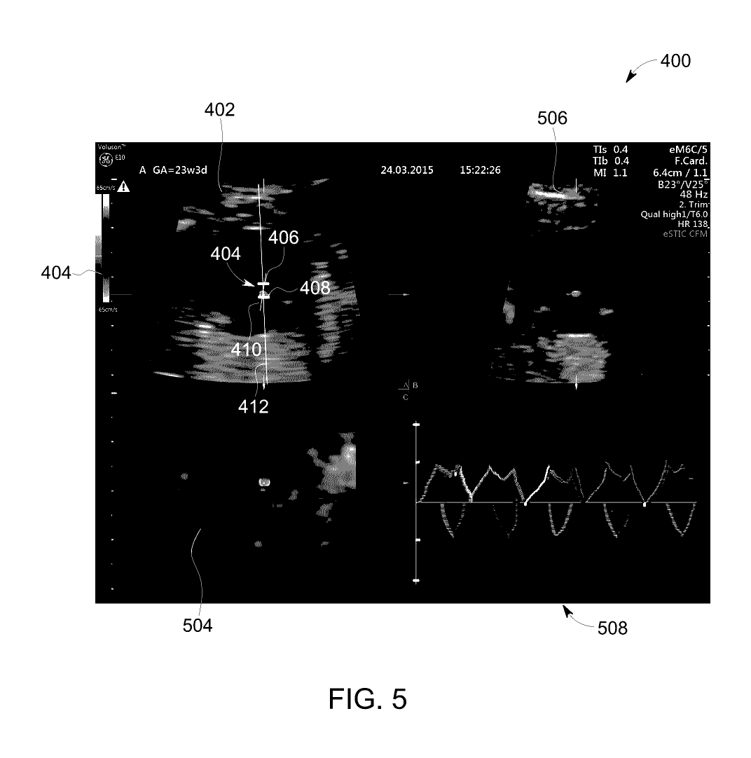

The scan converter 292, or generally the controller circuit 136, may generate the color flow image frames (e.g., 293) associated with the color flow data 273, and display the color flow image frames successively on the display 138 (e.g., as a cine loop). Additionally or alternatively, in connection with FIG. 5, the controller circuit 136 may display additional color flow images 504 and 506 of the ROI, concurrently.

FIG. 5 illustrates multiple color flow images 402 and 504-506 of the ROI, which may be shown on the display 138. Each of the color flow images 402 and 504-506 may correspond to an orthogonal plane of the ROI. For example, the color flow images 402 to a sagittal plane of the ROI, the second color flow image 504 to a traverse plane of the ROI, and the third color flow image 506 to a coronal plane of the ROI.

At 306, the controller circuit 136 designates a spatial gate 404 on the color flow image 402 corresponding to a set of voxels. For example, the controller circuit 136 may receive a user selection via the user interface 142 corresponding to a position of the spatial gate 404 within the color flow image 402. Additionally or alternatively, the spatial gate 404 may be positioned automatically by the controller circuit 136. The spatial gate 404 is illustrated as a cursor defined by a first and second border 406-408. For example, a size of the spatial gate 404 is represented by a distance between the first and second border 406-408. It should be noted that in other embodiments, the spatial gate 404 may have a different geometry or shape, such as a circle, trapezoid, and/or the like. Optionally, the user may adjust a size of the spatial gate 404 by adjust a position of the first border 406 and/or the second border 408. The spatial gate 404 corresponds to a set of voxels adjacent to one another and within the color flow image 402. For example, the spatial gate 404 may form a volume defined by the first and second border 406-408. The set of voxels are positioned within the volume of the spatial gate 404.

Optionally, the controller circuit 136 may display a beam centerline 412 and a slope cursor 410 with the spatial gate 404. The beam centerline 412 and the slope cursor 410 may form an angle value (e.g., Doppler angle), which may be used by the controller circuit 136 to convert the Doppler data, specifically the phase shifts, into a velocity.

At 308, the controller circuit 136 identifies a select time within the period of time. The select time may correspond to a set of ultrasound data corresponding to one or more of the frames acquired during the period of time. For example, during the period of time the controller circuit 136 may collect ultrasound data corresponding to a plurality of color flow image frames. The controller circuit 136 may select one of the color flow image frames, which define the select time within the period of time. For example, the controller circuit 136 may identify the color flow data 273 at the select time, such as the corresponding color flow image frame, stored on the memory 290.

Additionally or alternatively, the user may select one of the color flow image frames corresponding to the select time. For example, the controller circuit 136 may display a cine loop of the color flow image frames acquired during the period of time. The controller circuit 136 may receive an input from the user to stop the cine loop by selecting a user interface component and/or select keystroke using the user interface 142.

At 310, the controller circuit 136 calculates a mean velocity associated with the set of voxels at the select time. The mean velocity may be determined from the Doppler data. For example, the controller circuit 136 may identify the color flow data 273 at the select time, such as the corresponding color flow image frame, stored in the memory 290. The color flow data 273 includes the velocity data for each voxel of the color flow image frame, which was calculated from the Doppler data by the color flow circuit 252 or generally the controller circuit 136. The controller circuit 136 may identify select velocity data corresponding to the voxels within the set of voxels, and calculate a mean (e.g., average, arithmetic mean, geometric mean, harmonic mean) of the select velocity data.

Additionally or alternatively, the controller circuit 136 may calculate distinct mean velocities corresponding to an inflow mean velocity and an outflow mean velocity. For example, the select velocity data may include a direction of the velocity with respect to the ultrasound prove 126 corresponding to an inflow or an outflow. Generally, velocity data corresponding to an inflow direction may have a positive magnitude, and velocity data corresponding to an outflow direction may have a negative magnitude. The controller circuit 136 may calculate the inflow mean velocity by calculating a mean of the select velocity data having a positive magnitude, and the outflow velocity by calculating a mean of the select velocity data having a negative magnitude. It should be noted, the velocity magnitude may change in various other embodiments based on a positioned of the ultrasound probe 126 during acquisition of the ultrasound data.

At 312, the controller circuit 136 adds the mean velocity to a series of mean velocities. The series of mean velocities may be a collection of mean velocities, which are calculated at 310, with corresponding select times. The series of mean velocities may be stored in the memory, such has the memory 140 or the memory 290. Optionally, the series of mean velocities may include the inflow mean velocity and the outflow mean velocity for each corresponding select time.

At 314, the controller circuit 136 determines if additional select time may be selected to calculate a mean velocity, for example, at 310. In various embodiments, the controller circuit 136 may repeat the calculation operation at 310 for multiple select times over the period of time to derive the series of mean velocities. For example, when the controller circuit 136 determines that a mean velocity has not been calculated for each the color flow image frames, the controller circuit 136 may determine an additional select time may be selected and selects one of the remaining or alternative select times within the period of time at 316 and return to 310.

In another example, the controller circuit 136 may receive multiple select times within the time period by the user from the user interface 142. The controller circuit 136 may determine an additional select time may be selected from the multiple select times when a mean velocity has not been calculated for each of the multiple select times. The controller circuit 136 may select one of the remaining select times at 316 and return to 310.

If the controller circuit 136 determines no additional select times, at 318, the controller circuit presents indicia indicative of the series of mean velocities exhibited by the set of voxels within the spatial gate over the period of time. In various embodiments, the indicia may correspond to a table listing the set of mean velocities. Optionally, the indicia may correspond to a numerical value displayed concurrently with the color flow image 402. For example, the indicia may display a numeral value of a select mean velocity from the series of mean velocities corresponding to the select time (e.g., the color flow image frame) shown on the display 138. In connection with FIG. 5, the indicia may correspond to a graphical waveform 508 plotted over the select times, shown on the display 138. For example, the graphical waveform 508 may be shown concurrently with one or more of the color flow images 402 and 504-506.

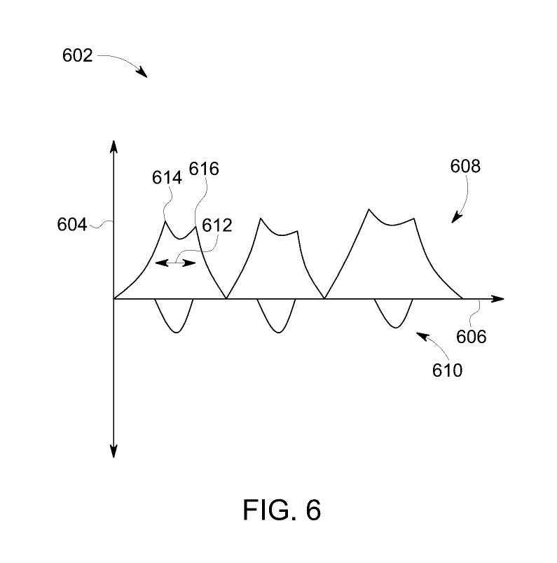

Additionally or alternatively, in connection with FIG. 6, an indicia 602 may include a first graphical waveform 608 corresponding to the inflow mean velocity and a second graphical waveform 610 corresponding to the outflow mean velocity.

FIG. 6 illustrates the indicia 602 of the series of mean velocities exhibited by the set of voxels. The first and second graphical waveforms 608 and 610 are plotted along a vertical axis 604 representing a velocity over time with respect to the time period represented by a horizontal axis 606.

At 320, the controller circuit 136 may determine abnormalities of the series of mean velocities. Abnormalities of the series of mean velocities may be determined by the controller circuit 136 based on changes of the mean velocity between select times within the time period. For example, the controller circuit 136 may determine abnormalities based on a morphology of the graphical waveform. The morphology may correspond to a peak amplitude, a number of peaks, peak width, peak latency, descending and/or ascending slopes, and/or the like. The morphology of the graphical waveform may be determined by the controller circuit 136, for example, based on changes in the graphical waveform over time. In connection with FIG. 6, the controller circuit 136 may determine peak velocities based on a magnitude or vertex of one or more peaks of the first graphical waveform 608.

The peak velocities may correspond to a phase of the cardiac cycle occurring during the time period, of which, at least a portion is represented by the first graphical waveform 608. For example, the controller circuit 136 may determine a peak systolic velocity or when the systole phase of the cardiac cycle occurs by identifying vertices of peaks. The controller circuit 136 may determine when a peak occurs based on changes in slope magnitudes corresponding to a vertex. For example, a change from a positive slope to a negative slope of the first graphical waveform 608 may indicate a peak, such as a first peak 614. Additionally or alternatively, the controller circuit 136 may take a derivative of the first graphical waveform 608 to determine vertexes of peaks, which have a value of zero.

The controller circuit 136 may compare the morphology of the graphical waveform with a graphical template to determine whether the series of mean velocities include abnormalities. The graphical template may be based on a position of the spatial gate 404 on the color flow image 402. For example, cardiac structures may have a unique flow pattern represented by different velocity directions corresponding to a morphology. A collection of candidate graphical templates with corresponding cardiac structures may be stored in the memory 140 and/or the memory 290. Based on a position of the spatial gate 404, the controller circuit 136 may select one of the graphical templates to compare with the morphology of the graphical waveform from the series of mean velocities. Each graphical template may include one or more morphology specifications and/or guidelines corresponding to the cardiac structure. For example, the controller circuit 136 may determine that a morphology of a graphical waveform that does not match the graphical template of the corresponding cardiac structure within a tolerance threshold includes abnormalities.

For example, the spatial gate 404 may be positioned within a left ventricle corresponding to the first and second graphical waveforms 608 and 610. The controller circuit 136 selects a template morphology from the candidate graphical templates stored in the memory 140 corresponding to the left ventricle. The graphical template of the left ventricle may define an M-shape inflow morphology having two peaks of similar magnitudes separated by a non-zero inflow. The controller circuit 136 may determine a first peak 614 and a second peak 616 of the first graphical waveform 608 corresponding to the inflow mean velocities. Based on a position of the first and second peak 614 and 616, the controller circuit 136 may define a transition region 612 between the first and second peak 614 and 616. The controller circuit 136 may compare the position of the mean velocity value during the transition region 612 with the horizontal axis 606 to determine that the first graphical waveform 608 does not have a non-zero inflow. Since the first graphical waveform 608 has an M-shape inflow based on the first and second peaks 614 and 616 separated by a non-zero inflow (e.g., the transition region 612), the controller circuit 136 may determine that the series of mean velocities does not include abnormalities.

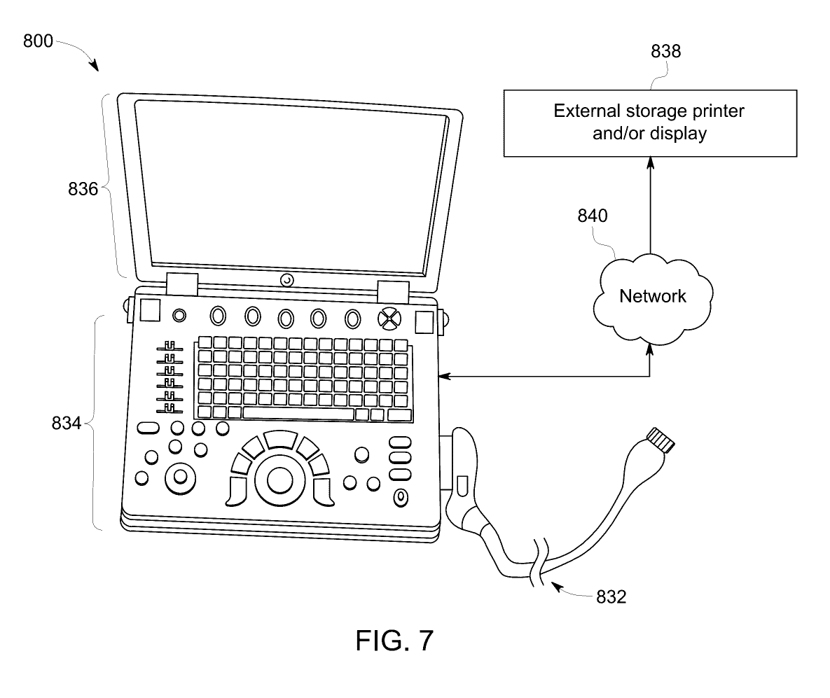

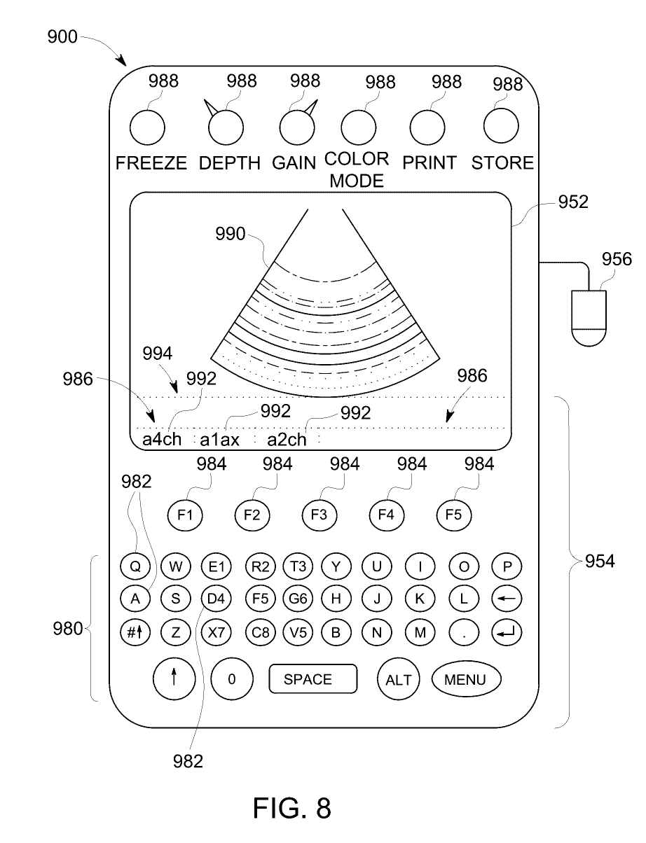

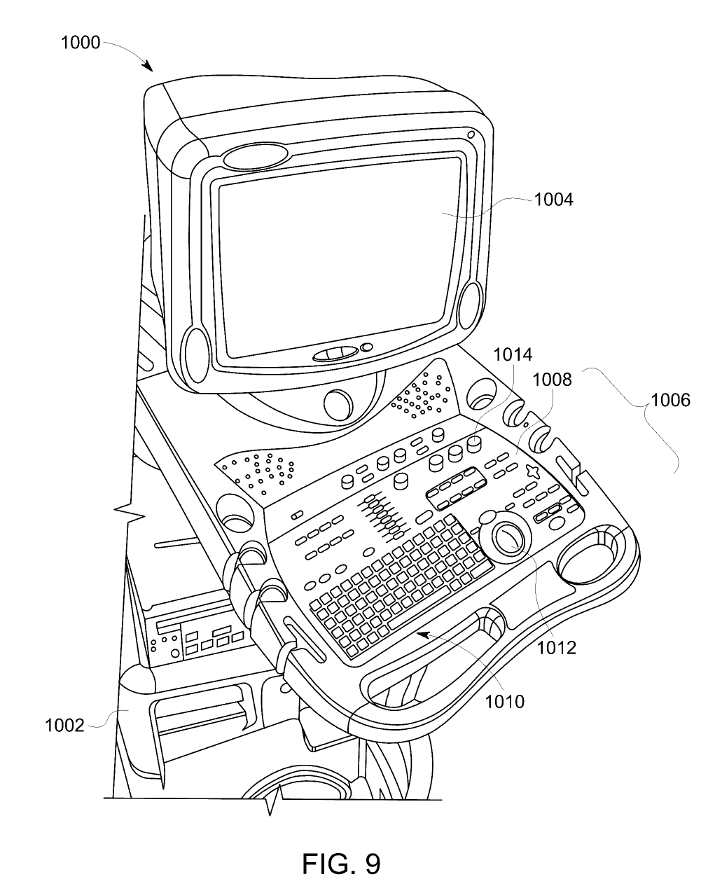

The ultrasound system 100 of FIG. 1 may be embodied in a small-sized system, such as laptop computer or pocket-sized system as well as in a larger console-type system. FIGS. 7 and 8 illustrate small-sized systems, while FIG. 9 illustrates a larger system.

FIG. 7 illustrates a 3D-capable miniaturized ultrasound system 800 having a probe 832 that may be configured to acquire 3D ultrasonic data or multi-plane ultrasonic data. For example, the probe 832 may have a 2D array of elements as discussed previously with respect to the probe. A user interface 834 (that may also include an integrated display 836) is provided to receive commands from an operator. As used herein, “miniaturized” means that the ultrasound system 800 is a handheld or hand-carried device or is configured to be carried in a person's hand, pocket, briefcase-sized case, or backpack. For example, the ultrasound system 800 may be a hand-carried device having a size of a typical laptop computer. The ultrasound system 800 is easily portable by the operator. The integrated display 836 (e.g., an internal display) is configured to display, for example, one or more medical images.

The ultrasonic data may be sent to an external device 838 via a wired or wireless network 840 (or direct connection, for example, via a serial or parallel cable or USB port). In some embodiments, the external device 838 may be a computer or a workstation having a display. Alternatively, the external device 838 may be a separate external display or a printer capable of receiving image data from the hand carried ultrasound system 800 and of displaying or printing images that may have greater resolution than the integrated display 836.

FIG. 8 illustrates a hand carried or pocket-sized ultrasound imaging system 900 wherein the display 952 and user interface 954 form a single unit. By way of example, the pocket-sized ultrasound imaging system 900 may be a pocket-sized or hand-sized ultrasound system approximately 2 inches wide, approximately 4 inches in length, and approximately 0.5 inches in depth and weighs less than 3 ounces. The pocket-sized ultrasound imaging system 900 generally includes the display 952, user interface 954, which may or may not include a keyboard-type interface and an input/output (I/O) port for connection to a scanning device, for example, an ultrasound probe 956. The display 952 may be, for example, a 320×320 pixel color LCD display (on which a medical image 990 may be displayed). A typewriter-like keyboard 980 of buttons 982 may optionally be included in the user interface 954.

Multi-function controls 984 may each be assigned functions in accordance with the mode of system operation (e.g., displaying different views). Therefore, each of the multi-function controls 984 may be configured to provide a plurality of different actions. One or more interface components, such as label display areas 986 associated with the multi-function controls 984 may be included as necessary on the display 952. The system 900 may also have additional keys and/or controls 988 for special purpose functions, which may include, but are not limited to “freeze,” “depth control,” “gain control,” “color-mode,” “print,” and “store.”

One or more of the label display areas 986 may include labels 992 to indicate the view being displayed or allow a user to select a different view of the imaged object to display. The selection of different views also may be provided through the associated multi-function control 984. The display 952 may also have one or more interface components corresponding to a textual display area 994 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 900 and the miniaturized ultrasound system 800 may provide the same scanning and processing functionality as the system 100.

FIG. 9 illustrates an ultrasound imaging system 1000 provided on a movable base 1002. The portable ultrasound imaging system 1000 may also be referred to as a cart-based system. A display 1004 and user interface 1006 are provided and it should be understood that the display 1004 may be separate or separable from the user interface 1006. The user interface 1006 may optionally be a touchscreen, allowing the operator to select options by touching displayed graphics, icons, and the like.

The user interface 1006 also includes control buttons 1008 that may be used to control the portable ultrasound imaging system 1000 as desired or needed, and/or as typically provided. The user interface 1006 provides multiple interface options that the user may physically manipulate to interact with ultrasound data and other data that may be displayed, as well as to input information and set and change scanning parameters and viewing angles, etc. For example, a keyboard 1010, trackball 1012 and/or multi-function controls 1014 may be provided.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid-state drive, optical disk drive, and the like. The storage device may also be other similar means for loading computer programs or other instructions into the computer or processor.

As used herein, the term “computer,” “subsystem” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein. Instead, the use of “configured to” as used herein denotes structural adaptations or characteristics, and denotes structural requirements of any structure, limitation, or element that is described as being “configured to” perform the task or operation. For example, a controller circuit, processor, or computer that is “configured to” perform a task or operation may be understood as being particularly structured to perform the task or operation (e.g., having one or more programs or instructions stored thereon or used in conjunction therewith tailored or intended to perform the task or operation, and/or having an arrangement of processing circuitry tailored or intended to perform the task or operation). For the purposes of clarity and the avoidance of doubt, a general purpose computer (which may become “configured to” perform the task or operation if appropriately programmed) is not “configured to” perform a task or operation unless or until specifically programmed or structurally modified to perform the task or operation.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal language of the claims.

What is claimed is:

1. A method for providing a mean velocity for ultrasound data, the method comprising:
collecting ultrasound data over a period of time for a volumetric region of interest (ROI);
displaying a color flow image based on the ultrasound data;
designating a spatial gate on the color flow image, the spatial gate corresponding to a set of voxels adjacent to one another and within the color flow image;
calculating a mean velocity associated with the set of voxels at a select time;
repeating the calculating operation for multiple select times over the period of time to derive a series of mean velocities; and
presenting indicia indicative of the series of mean velocities exhibited by the set of voxels within the spatial gate over the period of time.

2. The method of claim 1, wherein the indicia corresponds to a graphical waveform shown on a display.

3. The method of claim 2, further comprising:
determining a morphology of the graphical waveform; and
comparing the morphology with a graphical template, wherein the graphical template is based on a position of the spatial gate on the color flow image.

4. The method of claim 1, wherein the series of mean velocities include an inflow mean velocity and an outflow mean velocity, the indicia including a first graphical waveform corresponding to the inflow mean velocity and a second graphical waveform corresponding to the outflow mean velocity.

5. The method of claim 1, further comprising receiving a user selection corresponding to a position of the spatial gate within the color flow image.

6. The method of claim 1, wherein ultrasound data includes Doppler data, the mean velocity being determined from the Doppler data.

7. The method of claim 1, further comprising displaying second and third color flow images based on the ultrasound data, wherein the color image, the second color image, and third color flow image correspond to different planes of the ROI.

8. An ultrasound imaging system for a mean velocity comprising:
an ultrasound probe configured to acquire ultrasound data of a patient;
a display;
a memory configured to store programmed instructions; and
one or more processors configured to execute the programmed instructions stored in the memory, wherein the one or more processors when executing the programmed instructions perform the following operations:
collect the ultrasound data from the ultrasound probe over a period of time for a volumetric region of interest (ROI);
display a color flow image on the display, wherein the color flow image is based on the ultrasound data;
designate a spatial gate on the color flow image, the spatial gate corresponding to a set of voxels adjacent to one another and within the color flow image;
calculate a mean velocity associated with the set of voxels at a select time;
repeat the calculating operation for multiple select times over the period of time to derive a series of mean velocities; and
present indicia indicative of the series of mean velocities exhibited by the set of voxels within the spatial gate over the period of time.

9. The ultrasound imaging system of claim 8, wherein the indicia corresponds to a graphical waveform shown on a display.

10. The ultrasound imaging system of claim 9, wherein the one or more processors further determine a morphology of the graphical waveform, and compare the morphology with a graphical template, wherein the graphical template is based on a position of the spatial gate on the color flow image.

11. The ultrasound imaging system of claim 8, wherein the series of mean velocities include an inflow mean velocity and an outflow mean velocity, the indicia including a first graphical waveform corresponding to the inflow mean velocity and a second graphical waveform corresponding to the outflow mean velocity.

12. The ultrasound imaging system of claim 8, further comprising a user interface, wherein the one or more processors further receive a user selection corresponding to a position of the spatial gate within the color flow image from the user interface.

13. The ultrasound imaging system of claim 8, wherein ultrasound data includes Doppler data, the mean velocity being determined from the Doppler data.

14. The ultrasound imaging system of claim 8, wherein the one or more processors further display on the display second and third color flow images based on the ultrasound data, wherein the color image, the second color image, and third color flow image correspond to different planes of a region of interest.

15. A tangible and non-transitory computer readable medium comprising one or more computer software modules configured to direct one or more processors to:
collect ultrasound data over a period of time for a volumetric region of interest (ROI);
display a color flow image based on the ultrasound data;
designate a spatial gate on the color flow image, the spatial gate corresponding to a set of voxels adjacent to one another and within the color flow image;
calculate a mean velocity associated with the set of voxels at a select time;
repeat the calculating operation for multiple select times over the period of time to derive a series of mean velocities; and
present indicia indicative of the series of mean velocities exhibited by the set of voxels within the spatial gate over the period of time.

16. The tangible and non-transitory computer readable medium of claim 15, wherein the indicia corresponds to a graphical waveform shown on a display.

17. The tangible and non-transitory computer readable medium of claim 16, wherein the one or more processors are further directed to determine a morphology of the graphical waveform, and compare the morphology with a graphical template, wherein the graphical template is based on a position of the spatial gate on the color flow image.

18. The tangible and non-transitory computer readable medium of claim 15, wherein the series of mean velocities include an inflow mean velocity and an outflow mean velocity, the indicia including a first graphical waveform corresponding to the inflow mean velocity and a second graphical waveform corresponding to the outflow mean velocity.

19. The tangible and non-transitory computer readable medium of claim 15, wherein the one or more processor are further directed to receive a user selection corresponding to a position of the spatial gate within the color flow image.

20. The tangible and non-transitory computer readable medium of claim 15, wherein ultrasound data includes Doppler data, the mean velocity being determined from the Doppler data.
